(19) United States
(12) Patent Application Publication
(10) Pub. No.: US 2026/0096859 A1
(43) Pub. Date: Apr. 9, 2026

(54) SYSTEMS AND METHODS FOR CONFIGURING SURGICAL SYSTEMS TO PERFORM PATIENT-SPECIFIC PROCEDURE WITH SURGEON PREFERENCES

(71) Applicants: SMITH & NEPHEW, INC., Memphis, TN (US); SMITH & NEPHEW ORTHOPAEDICS AG, Zug (CH); SMITH & NEPHEW ASIA PACIFIC PTE. LIMITED, Singapore (SG)

(72) Inventors: Elizabeth A. DUXBURY, Andover, MA (US); Alessandro NAVACCHIA, Andover, MA (US); Nathan A. NETRAVALI, Andover, MA (US)

(21) Appl. No.: 19/113,281

(22) PCT Filed: Sep. 27, 2023
(86) PCT No.: PCT/US2023/033855

§ 371 Date: Mar. 19, 2025

Related U.S. Application Data
(60) Provisional application No. 63/410,775, filed on Sep. 28, 2022.

(51) Int. Cl. A61B 34/00; A61B 34/10; A61B 34/30; G06N 20/00

(52) U.S. Cl. CPC A61B 34/25; A61B 34/10; A61B 34/30; G06N 20/00; A61B 2034/104; A61B 2034/105; A61B 2034/108; A61B 2034/252; A61B 2034/256; A61B 2034/258

(57) ABSTRACT

Systems and methods for configuring a surgical system for performing a surgical procedure on a patient. The systems can include, for one or more patient categories, display a plurality of biomechanical simulations indicating postoperative performance of an implant based on alignment algorithms and receive a selection of one of the plurality of biomechanical simulations. In connection with the performance of a surgical procedure, the systems can retrieve a selected alignment algorithm corresponding to a patient category of the plurality of patient categories with which the patient is associated and configure the surgical system according to the selected alignment algorithm.

### CROSS-REFERENCE TO RELATED APPLICATIONS

This application claims priority to U.S. Provisional Patent Application 63/410,775, titled “SYSTEMS AND METHODS FOR CONFIGURING SURGICAL SYSTEMS TO PERFORM PATIENT-SPECIFIC PROCEDURE WITH SURGEON PREFERENCES,” filed on Sep. 28, 2022, which is hereby incorporated by reference herein in its entirety.

### TECHNICAL FIELD

The present disclosure relates generally to methods, systems, and apparatuses related to a computer-assisted surgical system that includes various hardware and software components that work together to enhance surgical workflows. The disclosed techniques may be applied to, for example, shoulder, hip, and knee arthroplasties, as well as other surgical interventions such as arthroscopic procedures, spinal procedures, maxillofacial procedures, neuro-surgery procedures, rotator cuff procedures, ligament repair and replacement procedures.

### BACKGROUND

Robotic and computer-assisted surgical systems can increase the accuracy and precision of surgical procedures, particularly by providing more accurate implant positioning, range of motion, soft tissue balance, reduced risk of injury to soft tissues, fewer outliers, and quicker recovery and less pain. However, robotic and computer-assisted surgical systems can require a significant amount of configuration and setup time, which is both cumbersome and can be complex for surgeons to learn and configure to their preferences. Further, less experienced surgeons may not understand the differences between planning philosophies (e.g., kinematic vs. mechanical vs. anatomic planning techniques) and implementation for different implant types. Therefore, there is a need in the prior art for surgical systems that are adapted to explain to surgeons (i) the results of advanced settings and parameters in an easily digestible manner and (ii) how to assign a best initial configuration. Further, there is a need in the prior art for the surgeon's preferences to be automatically loaded into the surgical systems at the time of operation in order to minimize the amount of initial setup and pre-configuration that the surgical team must perform in the operating room.

### SUMMARY

The present disclosure is directed to systems and methods for planning patient-specific surgical procedures.

In some embodiments, the present disclosure is directed to a computer-implemented method for configuring a surgical system for performing a surgical procedure on a patient, the surgical procedure comprising a plurality of surgical parameters, the method comprising: for each of a plurality of patient categories: displaying a plurality of biomechanical simulations indicating postoperative performance of an implant, each of the plurality of biomechanical simulations corresponding to one of a plurality of alignment algorithms, and receiving, from a user, a selection of one of the plurality of biomechanical simulations, storing one of the plurality of alignment algorithms corresponding to each selection for each of the plurality of patient categories, thereby defining a user profile comprising a plurality of selected alignment algorithms corresponding to the plurality of patient categories. In response to the surgical system being used to perform the surgical procedure on the patient by the user the computer-implemented method includes retrieving, from the user profile, a selected alignment algorithm corresponding to a patient category of the plurality of patient categories with which the patient is associated, and configuring the surgical system according to the selected alignment algorithm.

In some embodiments, the plurality of alignment algorithms are selected from the group consisting of anatomic alignment, kinematic alignment, mechanical alignment, and physiological alignment.

In some embodiments, the plurality of alignment algorithms are manually configured.

In some embodiments, the plurality of alignment algorithms are clustered by a machine learning model based on historical patient data.

In some embodiments, the surgical procedure is selected from the group consisting of a knee arthroplasty, a hip arthroplasty, and a shoulder arthroplasty.

In some embodiments, the surgical system comprises a robotic surgical system.

In some embodiments, the plurality of patient categories correspond to at least one of full leg varus/valgus angle, flexion-contracture degree, or joint line obliquity.

In some embodiments, the method further comprises repeating the displaying of the plurality of biomechanical simulations indicating postoperative performance for a plurality of implant types; and receiving, from a user, the selection of one of the plurality of biomechanical simulations for each of the plurality of implant types.

In some embodiments, the present disclosure is directed to a system for performing a surgical procedure on a patient, the system including a computer system including a first processor and a first non-transitory memory. The first non-transitory memory stores first instructions that, when executed by the first processor, cause the computer system to for each of a plurality of patient categories: display a plurality of biomechanical simulations indicating postoperative performance of an implant, each of the plurality of biomechanical simulations corresponding to one of a plurality of alignment algorithms, and receive, from a user, a selection of one of the plurality of biomechanical simulations, store one of the plurality of alignment algorithms corresponding to each selection for each of the plurality of patient categories, thereby defining a user profile comprising a plurality of selected alignment algorithms corresponding to the plurality of patient categories. The system further includes a surgical system communicatively coupled to the computer system, the surgical system includes a second processor and a second memory. The second non-transitory memory stores second instructions that, when executed by the second processor, cause the surgical system to: in response to the surgical system being used to perform the surgical procedure on a patient by the user: retrieve, from the user profile, a selected alignment algorithm corresponding to a patient category of the plurality of patient categories with which the patient is associated, and configure the surgical system according to the selected alignment algorithm.

In some embodiments, the plurality of alignment algorithms are selected from the group consisting of anatomic alignment, kinematic alignment, mechanical alignment, and physiological alignment.

In some embodiments, the plurality of alignment algorithms are manually configured.

In some embodiments, the plurality of alignment algorithms are clustered by a machine learning model based on historical patient data

In some embodiments, the surgical procedure is selected from the group consisting of a knee arthroplasty, a hip arthroplasty, and a shoulder arthroplasty.

In some embodiments, the surgical system includes a robotic surgical system.

In some embodiments, the plurality of patient categories correspond to at least one of full leg varus/valgus angle, flexion-contracture degree, or joint line obliquity.

In some embodiments, the first instructions, when executed by the first processor, further cause the computer system to repeat the displaying of the plurality of biomechanical simulations indicating postoperative performance for a plurality of implant types, and receive, from a user, the selection of one of the plurality of biomechanical simulations for each of the plurality of implant types.

In some embodiments, the present disclosure is directed to system for performing a surgical procedure on a patient, the system including a computer system comprising a first processor and a first non-transitory memory. The first non-transitory memory store first instructions that, when executed by the first processor, cause the computer system to train a machine learning model on a training set of patient outcome data; cluster, by the machine learning model, a plurality of alignment algorithms; for each of a plurality of patient categories: display a plurality of biomechanical simulations indicating postoperative performance of an implant, each of the plurality of biomechanical simulations corresponding to one of the plurality of alignment algorithms, and receive, from a user, a selection of one of the plurality of biomechanical simulations, store one of the plurality of alignment algorithms corresponding to each selection for each of the plurality of patient categories, thereby defining a user profile comprising a plurality of selected alignment algorithms corresponding to the plurality of patient categories. The system further includes a surgical system communicatively coupled to the computer system, the surgical system comprising a second processor and a second non-transitory memory. The second non-transitory memory stores second instructions that, when executed by the second processor, cause the surgical system to in response to the surgical system being used to perform the surgical procedure on a patient by the user retrieve, from the user profile, a selected alignment algorithm corresponding to a patient category of the plurality of patient categories with which the patient is associated, and configure the surgical system according to the selected alignment algorithm.

In some embodiments, the machine learning model is supervised.

In some embodiments, the machine learning model is unsupervised.

In some embodiments, the system further comprises a robotic surgical component, wherein the second instructions, when executed by the second processor, further cause the surgical system to navigate the robotic surgical component according to the selected alignment algorithm.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1 depicts an operating theatre including an illustrative computer-assisted surgical system (CASS) in accordance with an embodiment.

FIG. 2A depicts illustrative control instructions that a surgical computer provides to other components of a CASS in accordance with an embodiment.

FIG. 2B depicts illustrative control instructions that components of a CASS provide to a surgical computer in accordance with an embodiment.

FIG. 2C depicts an illustrative implementation in which a surgical computer is connected to a surgical data server via a network in accordance with an embodiment.

FIG. 3 depicts a diagram of a system in accordance with an embodiment.

FIG. 4 depicts a logic flow diagram of a process for planning a patient-specific surgical procedure in accordance with an embodiment.

FIG. 5 depicts a graphical user interface showing full leg alignment patient categories in accordance with an embodiment.

FIG. 6A depicts a graphical user interface showing a biomechanical simulation for a selected patient category in accordance with an embodiment.

FIG. 6B depicts a graphical user interface showing a comparison of multiple biomechanical simulations for a selected patient category in accordance with an embodiment.

FIG. 7 depicts a graphical user interface showing both manually derived and AI-derived alignment algorithms in accordance with an embodiment.

### DETAILED DESCRIPTION

For the purposes of this disclosure, the term “implant” is used to refer to a prosthetic device or structure manufactured to replace or enhance a biological structure. For example, in a total hip replacement procedure a prosthetic acetabular cup (implant) is used to replace or enhance a patients worn or damaged acetabulum. While the term “implant” is generally considered to denote a man-made structure (as contrasted with a transplant), for the purposes of this specification an implant can include a biological tissue or material transplanted to replace or enhance a biological structure.

For the purposes of this disclosure, the term “real-time” is used to refer to calculations or operations performed on-the-fly as events occur or input is received by the operable system. However, the use of the term “real-time” is not intended to preclude operations that cause some latency between input and response, so long as the latency is an unintended consequence induced by the performance characteristics of the machine.

Although much of this disclosure refers to surgeons or other medical professionals by specific job title or role, nothing in this disclosure is intended to be limited to a specific job title or function. Surgeons or medical professionals can include any doctor, nurse, medical professional, or technician. Any of these terms or job titles can be used interchangeably with the user of the systems disclosed herein unless otherwise explicitly demarcated. For example, a reference to a surgeon also could apply, in some embodiments to a technician or nurse.

The systems, methods, and devices disclosed herein are particularly well adapted for surgical procedures that utilize surgical navigation systems, such as the CORI® surgical navigation system. CORI is a registered trademark of BLUE BELT TECHNOLOGIES, INC. of Pittsburgh, PA, which is a subsidiary of SMITH & NEPHEW, INC. of Memphis, TN.

### CASS Ecosystem Overview

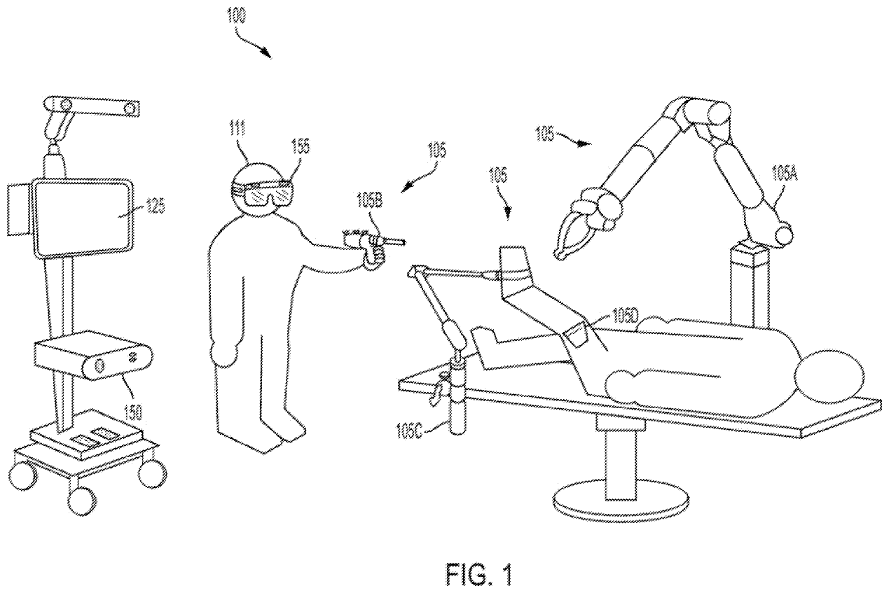

FIG. 1 provides an illustration of an example computer-assisted surgical system (CASS) 100, according to some embodiments. As described in further detail in the sections that follow, the CASS uses computers, robotics, and imaging technology to aid surgeons in performing orthopedic surgery procedures such as total knee arthroplasty (TKA) or THA. For example, surgical navigation systems can aid surgeons in locating patient anatomical structures, guiding surgical instruments, and implanting medical devices with a high degree of accuracy. Surgical navigation systems such as the CASS 100 often employ various forms of computing technology to perform a wide variety of standard and minimally invasive surgical procedures and techniques. Moreover, these systems allow surgeons to more accurately plan, track and navigate the placement of instruments and implants relative to the body of a patient, as well as conduct pre-operative and intra-operative body imaging.

An Effector Platform 105 positions surgical tools relative to a patient during surgery. The exact components of the Effector Platform 105 will vary, depending on the embodiment employed. For example, for a knee surgery, the Effector Platform 105 may include an End Effector 105B that holds surgical tools or instruments during their use. The End Effector 105B may be a handheld device or instrument used by the surgeon (e.g., a CORI® hand piece or a cutting guide or jig) or, alternatively, the End Effector 105B can include a device or instrument held or positioned by a robotic arm 105A. While one robotic arm 105A is illustrated in FIG. 1, in some embodiments there may be multiple devices. As examples, there may be one robotic arm 105A on each side of an operating table T or two devices on one side of the table T. The robotic arm 105A may be mounted directly to the table T, be located next to the table T on a floor platform (not shown), mounted on a floor-to-ceiling pole, or mounted on a wall or ceiling of an operating room. The floor platform may be fixed or moveable. In one particular embodiment, the robotic arm 105A is mounted on a floor-to-ceiling pole located between the patient's legs or feet. In some embodiments, the End Effector 105B may include a suture holder or a stapler to assist in closing wounds. Further, in the case of two robotic arms 105A, the surgical computer 150 can drive the robotic arms 105A to work together to suture the wound at closure. Alternatively, the surgical computer 150 can drive one or more robotic arms 105A to staple the wound at closure.

The Effector Platform 105 can include a Limb Positioner 105C for positioning the patient's limbs during surgery. One example of a Limb Positioner 105C is the SMITH AND NEPHEW SPIDER2 system. The Limb Positioner 105C may be operated manually by the surgeon or alternatively change limb positions based on instructions received from the Surgical Computer 150 (described below). While one Limb Positioner 105C is illustrated in FIG. 1, in some embodiments there may be multiple devices. As examples, there may be one Limb Positioner 105C on each side of the operating table T or two devices on one side of the table T. The Limb Positioner 105C may be mounted directly to the table T, be located next to the table T on a floor platform (not shown), mounted on a pole, or mounted on a wall or ceiling of an operating room. In some embodiments, the Limb Positioner 105C can be used in non-conventional ways, such as a retractor or specific bone holder. The Limb Positioner 105° C. may include, as examples, an ankle boot, a soft tissue clamp, a bone clamp, or a soft-tissue retractor spoon, such as a hooked, curved, or angled blade. In some embodiments, the Limb Positioner 105C may include a suture holder to assist in closing wounds.

The Effector Platform 105 may include tools, such as a screwdriver, light or laser, to indicate an axis or plane, bubble level, pin driver, pin puller, plane checker, pointer, finger, or some combination thereof.

Resection Equipment 110 (not shown in FIG. 1) performs bone or tissue resection using, for example, mechanical, ultrasonic, or laser techniques. Examples of Resection Equipment 110 include drilling devices, burring devices, oscillatory sawing devices, vibratory impaction devices, reamers, ultrasonic bone cutting devices, radio frequency ablation devices, reciprocating devices (such as a rasp or broach), and laser ablation systems. In some embodiments, the Resection Equipment 110 is held and operated by the surgeon during surgery. In other embodiments, the Effector Platform 105 may be used to hold the Resection Equipment 110 during use.

The Effector Platform 105 also can include a cutting guide or jig 105D that is used to guide saws or drills used to resect tissue during surgery. Such cutting guides 10SD can be formed integrally as part of the Effector Platform 105 or robotic arm 105A, or cutting guides can be separate structures that can be matingly and/or removably attached to the Effector Platform 105 or robotic arm 105A. The Effector Platform 105 or robotic arm 105A can be controlled by the CASS 100 to position a cutting guide or jig 105D adjacent to the patient's anatomy in accordance with a pre-operatively or intraoperatively developed surgical plan such that the cutting guide or jig will produce a precise bone cut in accordance with the surgical plan.

The Tracking System 115 uses one or more sensors to collect real-time position data that locates the patient's anatomy and surgical instruments. For example, for TKA procedures, the Tracking System may provide a location and orientation of the End Effector 105B during the procedure. In addition to positional data, data from the Tracking System 115 also can be used to infer velocity/acceleration of anatomy/instrumentation, which can be used for tool control. In some embodiments, the Tracking System 115 may use a tracker array attached to the End Effector 105B to determine the location and orientation of the End Effector 105B. The position of the End Effector 105B may be inferred based on the position and orientation of the Tracking System 115 and a known relationship in three-dimensional space between the Tracking System 115 and the End Effector 105B. Various types of tracking systems may be used in various embodiments of the present invention including, without limitation, Infrared (IR) tracking systems, electromagnetic (EM) tracking systems. video or image based tracking systems, and ultrasound registration and tracking systems. Using the data provided by the tracking system 115, the surgical computer 150 can detect objects and prevent collision. For example, the surgical computer 150 can prevent the robotic arm 105A and/or the End Effector 105B from colliding with soft tissue.

Any suitable tracking system can be used for tracking surgical objects and patient anatomy in the surgical theatre. For example, a combination of IR and visible light cameras can be used in an array. Various illumination sources, such as an IR LED light source, can illuminate the scene allowing three-dimensional imaging to occur. In some embodiments. this can include stereoscopic, tri-scopic, quad-scopic, etc. imaging. In addition to the camera array, which in some embodiments is affixed to a cart, additional cameras can be placed throughout the surgical theatre. For example, handheld tools or headsets worn by operators/surgeons can include imaging capability that communicates images back to a central processor to correlate those images with images captured by the camera array. This can give a more robust image of the environment for modeling using multiple perspectives. Furthermore, some imaging devices may be of suitable resolution or have a suitable perspective on the scene to pick up information stored in quick response (QR) codes or barcodes. This can be helpful in identifying specific objects not manually registered with the system. In some embodiments, the camera may be mounted on the robotic arm 105A.

In some embodiments, specific objects can be manually registered by a surgeon with the system preoperatively or intraoperatively. For example, by interacting with a user interface, a surgeon may identify the starting location for a tool or a bone structure. By tracking fiducial marks associated with that tool or bone structure, or by using other conventional image tracking modalities, a processor may track that tool or bone as it moves through the environment in a three-dimensional model.

In some embodiments, certain markers, such as fiducial marks that identify individuals, important tools, or bones in the theater may include passive or active identifiers that can be picked up by a camera or camera array associated with the tracking system. For example, an IR LED can flash a pattern that conveys a unique identifier to the source of that pattern, providing a dynamic identification mark. Similarly, one or two dimensional optical codes (barcode, QR code, etc.) can be affixed to objects in the theater to provide passive identification that can occur based on image analysis. If these codes are placed asymmetrically on an object, they also can be used to determine an orientation of an object by comparing the location of the identifier with the extents of an object in an image. For example, a QR code may be placed in a corner of a tool tray, allowing the orientation and identity of that tray to be tracked. Other tracking modalities are explained throughout. For example, in some embodiments, augmented reality (AR) headsets can be worn by surgeons and other staff to provide additional camera angles and tracking capabilities. In this case, the infrared/time of flight sensor data, which is predominantly used for hand/gesture detection, can build correspondence between the AR headset and the tracking system of the robotic system using sensor fusion techniques. This can be used to calculate a calibration matrix that relates the optical camera coordinate frame to the fixed holographic world frame.

In addition to optical tracking, certain features of objects can be tracked by registering physical properties of the object and associating them with objects that can be tracked, such as fiducial marks fixed to a tool or bone. For example, a surgeon may perform a manual registration process whereby a tracked tool and a tracked bone can be manipulated relative to one another. By impinging the tip of the tool against the surface of the bone, a three-dimensional surface can be mapped for that bone that is associated with a position and orientation relative to the frame of reference of that fiducial mark. By optically tracking the position and orientation (pose) of the fiducial mark associated with that bone, a model of that surface can be tracked with an environment through extrapolation.

The registration process that registers the CASS 100 to the relevant anatomy of the patient also can involve the use of anatomical landmarks, such as landmarks on a bone or cartilage. For example, the CASS 100 can include a 3D model of the relevant bone or joint and the surgeon can intraoperatively collect data regarding the location of bony landmarks on the patient's actual bone using a probe that is connected to the CASS. Bony landmarks can include, for example, the medial malleolus and lateral malleolus, the ends of the proximal femur and distal tibia, and the center of the hip joint. The CASS 100 can compare and register the location data of bony landmarks collected by the surgeon with the probe with the location data of the same landmarks in the 3D model. Alternatively, the CASS 100 can construct a 3D model of the bone or joint without pre-operative image data by using location data of bony landmarks and the bone surface that are collected by the surgeon using a CASS probe or other means. The registration process also can include determining various axes of a joint. For example, for a TKA the surgeon can use the CASS 100 to determine the anatomical and mechanical axes of the femur and tibia. The surgeon and the CASS 100 can identify the center of the hip joint by moving the patient's leg in a spiral direction (i.e., circumduction) so the CASS can determine where the center of the hip joint is located.

A Tissue Navigation System 120 (not shown in FIG. 1) provides the surgeon with intraoperative, real-time visualization for the patient's bone, cartilage, muscle, nervous, and/or vascular tissues surrounding the surgical area. Examples of systems that may be employed for tissue navigation include fluorescent imaging systems and ultrasound systems.

The Display 125 provides graphical user interfaces (GUIs) that display images collected by the Tissue Navigation System 120 as well other information relevant to the surgery. For example, in one embodiment, the Display 125 overlays image information collected from various modalities (e.g., CT, MRI, X-ray, fluorescent, ultrasound, etc.) collected pre-operatively or intra-operatively to give the surgeon various views of the patient's anatomy as well as real-time conditions. The Display 125 may include, for example, one or more computer monitors. As an alternative or supplement to the Display 125, one or more members of the surgical staff may wear an Augmented Reality (AR) Head Mounted Device (HMD). For example, in FIG. 1 the Surgeon 111 is wearing an AR HMD 155 that may, for example, overlay pre-operative image data on the patient or provide surgical planning suggestions. In one embodiment, a tracker array-mounted surgical tool could be detected by both the IR camera and an AR headset (HMD) using sensor fusion techniques without the need for any “intermediate” calibration rigs. This near-depth, time-of-flight sensing camera located in the HMD could be used for hand/gesture detection. The headset's sensor API can be used to expose IR and depth image data and carryout image processing using, for example, C++ with OpenCV. This approach allows the relationship between the CASS and the virtual coordinate frame to be determined and the headset sensor data (i.e., IR in combination with depth images) to isolate the CASS tracker arrays. The image processing system on the HMD can locate the surgical tool in a fixed holographic world frame and the CASS IR camera can locate the surgical tool relative to its camera coordinate frame. This relationship can be used to calculate a calibration matrix that relates the CASS IR camera coordinate frame to the fixed holographic world frame. This means that if a calibration matrix has previously been calculated, the surgical tool no longer needs to be visible to the AR headset. However, a recalculation may be necessary if the CASS camera is accidentally moved in the workflow. Various example uses of the AR HMD 155 in surgical procedures are detailed in the sections that follow.

Surgical Computer 150 provides control instructions to various components of the CASS 100, collects data from those components, and provides general processing for various data needed during surgery. In some embodiments, the Surgical Computer 150 is a general purpose computer. In other embodiments, the Surgical Computer 150 may be a parallel computing platform that uses multiple central processing units (CPUs) or graphics processing units (GPU) to perform processing. In some embodiments, the Surgical Computer 150 is connected to a remote server over one or more computer networks (e.g., the Internet). The remote server can be used, for example, for storage of data or execution of computationally intensive processing tasks.

Various techniques generally known in the art can be used for connecting the Surgical Computer 150 to the other components of the CASS 100. Moreover, the computers can connect to the Surgical Computer 150 using a mix of technologies. For example, the End Effector 105B may connect to the Surgical Computer 150 over a wired (i.e., serial) connection. The Tracking System 115, Tissue Navigation System 120, and Display 125 can similarly be connected to the Surgical Computer 150 using wired connections. Alternatively, the Tracking System 115, Tissue Navigation System 120, and Display 125 may connect to the Surgical Computer 150 using wireless technologies such as, without limitation, Wi-Fi, Bluetooth, Near Field Communication (NFC), or ZigBee.

### Robotic Arm

In some embodiments, the CASS 100 includes a robotic arm 105A that serves as an interface to stabilize and hold a variety of instruments used during the surgical procedure. For example, in the context of a hip surgery, these instruments may include, without limitation, retractors, a sagittal or reciprocating saw, the reamer handle, the cup impactor, the broach handle, and the stem inserter. The robotic arm 105A may have multiple degrees of freedom (like a Spider device), and have the ability to be locked in place (e.g., by a press of a button, voice activation, a surgeon removing a hand from the robotic arm, or other method).

In some embodiments, movement of the robotic arm 105A may be effectuated by use of a control panel built into the robotic arm system. For example, a display screen may include one or more input sources, such as physical buttons or a user interface having one or more icons, that direct movement of the robotic arm 105A. The surgeon or other healthcare professional may engage with the one or more input sources to position the robotic arm 105A when performing a surgical procedure.

A tool or an end effector 105B attached or integrated into a robotic arm 105A may include, without limitation, a burring device, a scalpel, a cutting device, a retractor, a joint tensioning device, or the like. In embodiments in which an end effector 105B is used, the end effector may be positioned at the end of the robotic arm 105A such that any motor control operations are performed within the robotic arm system. In embodiments in which a tool is used, the tool may be secured at a distal end of the robotic arm 105A, but motor control operation may reside within the tool itself.

The robotic arm 105A may be motorized internally to both stabilize the robotic arm, thereby preventing it from falling and hitting the patient, surgical table, surgical staff, etc., and to allow the surgeon to move the robotic arm without having to fully support its weight. While the surgeon is moving the robotic arm 105A, the robotic arm may provide some resistance to prevent the robotic arm from moving too fast or having too many degrees of freedom active at once. The position and the lock status of the robotic arm 105A may be tracked, for example, by a controller or the Surgical Computer 150.

In some embodiments, the robotic arm 105A can be moved by hand (e.g., by the surgeon) or with internal motors into its ideal position and orientation for the task being performed. In some embodiments, the robotic arm 105A may be enabled to operate in a “free” mode that allows the surgeon to position the arm into a desired position without being restricted. While in the free mode, the position and orientation of the robotic arm 105A may still be tracked as described above. In one embodiment, certain degrees of freedom can be selectively released upon input from user (e.g., surgeon) during specified portions of the surgical plan tracked by the Surgical Computer 150. Designs in which a robotic arm 105A is internally powered through hydraulics or motors or provides resistance to external manual motion through similar means can be described as powered robotic arms, while arms that are manually manipulated without power feedback, but which may be manually or automatically locked in place, may be described as passive robotic arms.

A robotic arm 105A or end effector 105B can include a trigger or other means to control the power of a saw or drill. Engagement of the trigger or other means by the surgeon can cause the robotic arm 105A or end effector 105B to transition from a motorized alignment mode to a mode where the saw or drill is engaged and powered on. Additionally, the CASS 100 can include a foot pedal (not shown) that causes the system to perform certain functions when activated. For example, the surgeon can activate the foot pedal to instruct the CASS 100 to place the robotic arm 105A or end effector 105B in an automatic mode that brings the robotic arm or end effector into the proper position with respect to the patient's anatomy in order to perform the necessary resections. The CASS 100 also can place the robotic arm 105A or end effector 105B in a collaborative mode that allows the surgeon to manually manipulate and position the robotic arm or end effector into a particular location. The collaborative mode can be configured to allow the surgeon to move the robotic arm 105A or end effector 105B medially or laterally, while restricting movement in other directions. As discussed, the robotic arm 105A or end effector 105B can include a cutting device (saw, drill, and burr) or a cutting guide or jig 105D that will guide a cutting device. In other embodiments, movement of the robotic arm 105A or robotically controlled end effector 105B can be controlled entirely by the CASS 100 without any, or with only minimal, assistance or input from a surgeon or other medical professional. In still other embodiments, the movement of the robotic arm 105A or robotically controlled end effector 105B can be controlled remotely by a surgeon or other medical professional using a control mechanism separate from the robotic arm or robotically controlled end effector device, for example using a joystick or interactive monitor or display control device.

A robotic arm 105A may be used for holding the retractor. For example, in one embodiment, the robotic arm 105A may be moved into the desired position by the surgeon. At that point, the robotic arm 105A may lock into place. In some embodiments, the robotic arm 105A is provided with data regarding the patient's position, such that if the patient moves, the robotic arm can adjust the retractor position accordingly. In some embodiments, multiple robotic arms may be used, thereby allowing multiple retractors to be held or for more than one activity to be performed simultaneously (e.g., retractor holding & reaming).

The robotic arm 105A may also be used to help stabilize the surgeon's hand while making a femoral neck cut. In this application, control of the robotic arm 105A may impose certain restrictions to prevent soft tissue damage from occurring. For example, in one embodiment, the Surgical Computer 150 tracks the position of the robotic arm 105A as it operates. If the tracked location approaches an area where tissue damage is predicted, a command may be sent to the robotic arm 105A causing it to stop. Alternatively, where the robotic arm 105A is automatically controlled by the Surgical Computer 150, the Surgical Computer may ensure that the robotic arm is not provided with any instructions that cause it to enter areas where soft tissue damage is likely to occur. The Surgical Computer 150 may impose certain restrictions on the surgeon to prevent the surgeon from reaming too far into the medial wall of the acetabulum or reaming at an incorrect angle or orientation.

In some embodiments, the robotic arm 105A may be used to hold a cup impactor at a desired angle or orientation during cup impaction. When the final position has been achieved, the robotic arm 105A may prevent any further seating to prevent damage to the pelvis.

The surgeon may use the robotic arm 105A to position the broach handle at the desired position and allow the surgeon to impact the broach into the femoral canal at the desired orientation. In some embodiments, once the Surgical Computer 150 receives feedback that the broach is fully seated, the robotic arm 105A may restrict the handle to prevent further advancement of the broach.

The robotic arm 105A may also be used for resurfacing applications. For example, the robotic arm 105A may stabilize the surgeon while using traditional instrumentation and provide certain restrictions or limitations to allow for proper placement of implant components (e.g., guide wire placement, chamfer cutter, sleeve cutter, plan cutter, etc.). Where only a burr is employed, the robotic arm 105A may stabilize the surgeon's handpiece and may impose restrictions on the handpiece to prevent the surgeon from removing unintended bone in contravention of the surgical plan.

The robotic arm 105A may be a passive arm. As an example, the robotic arm 105A may be a CIRQ robot arm available from Brainlab AG. CIRQ is a registered trademark of Brainlab AG, Olof-Palme-Str. 9 81829, München, FED REP of GERMANY. In one particular embodiment, the robotic arm 105A is an intelligent holding arm as disclosed in U.S. patent application Ser. No. 15/525,585 to Krinninger et al., U.S. patent application Ser. No. 15/561,042 to Nowatschin et al., U.S. patent application Ser. No. 15/561,048 to Nowatschin et al., and U.S. Pat. No. 10,342,636 to Nowatschin et al., the entire contents of each of which is herein incorporated by reference.

### Surgical Procedure Data Generation and Collection

The various services that are provided by medical professionals to treat a clinical condition are collectively referred to as an “episode of care.” For a particular surgical intervention the episode of care can include three phases: pre-operative, intra-operative, and post-operative. During each phase, data is collected or generated that can be used to analyze the episode of care in order to understand various features of the procedure and identify patterns that may be used, for example, in training models to make decisions with minimal human intervention. The data collected over the episode of care may be stored at the Surgical Computer 150 or the Surgical Data Server 180 as a complete dataset. Thus, for each episode of care, a dataset exists that comprises all of the data collectively pre-operatively about the patient, all of the data collected or stored by the CASS 100 intra-operatively, and any post-operative data provided by the patient or by a healthcare professional monitoring the patient.

As explained in further detail, the data collected during the episode of care may be used to enhance performance of the surgical procedure or to provide a holistic understanding of the surgical procedure and the patient outcomes. For example, in some embodiments, the data collected over the episode of care may be used to generate a surgical plan. In one embodiment, a high-level, pre-operative plan is refined intra-operatively as data is collected during surgery. In this way, the surgical plan can be viewed as dynamically changing in real-time or near real-time as new data is collected by the components of the CASS 100. In other embodiments, pre-operative images or other input data may be used to develop a robust plan preoperatively that is simply executed during surgery. In this case, the data collected by the CASS 100 during surgery may be used to make recommendations that ensure that the surgeon stays within the pre-operative surgical plan. For example, if the surgeon is unsure how to achieve a certain prescribed cut or implant alignment, the Surgical Computer 150 can be queried for a recommendation. In still other embodiments, the pre-operative and intra-operative planning approaches can be combined such that a robust pre-operative plan can be dynamically modified, as necessary or desired, during the surgical procedure. In some embodiments, a biomechanics-based model of patient anatomy contributes simulation data to be considered by the CASS 100 in developing preoperative, intraoperative, and post-operative/rehabilitation procedures to optimize implant performance outcomes for the patient.

Aside from changing the surgical procedure itself, the data gathered during the episode of care may be used as an input to other procedures ancillary to the surgery. For example, in some embodiments, implants can be designed using episode of care data. Example data-driven techniques for designing, sizing, and fitting implants are described in U.S. Pat. No. 10,064,686, filed Aug. 15, 2011, and entitled “Systems and Methods for Optimizing Parameters for Orthopaedic Procedures”; U.S. Pat. No. 10,102,309, filed Jul. 20, 2012 and entitled “Systems and Methods for Optimizing Fit of an Implant to Anatomy”; and U.S. Pat. No. 8,078,440, filed Sep. 19, 2008 and entitled “Operatively Tuning Implants for Increased Performance,” the entire contents of each of which are hereby incorporated by reference into this patent application.

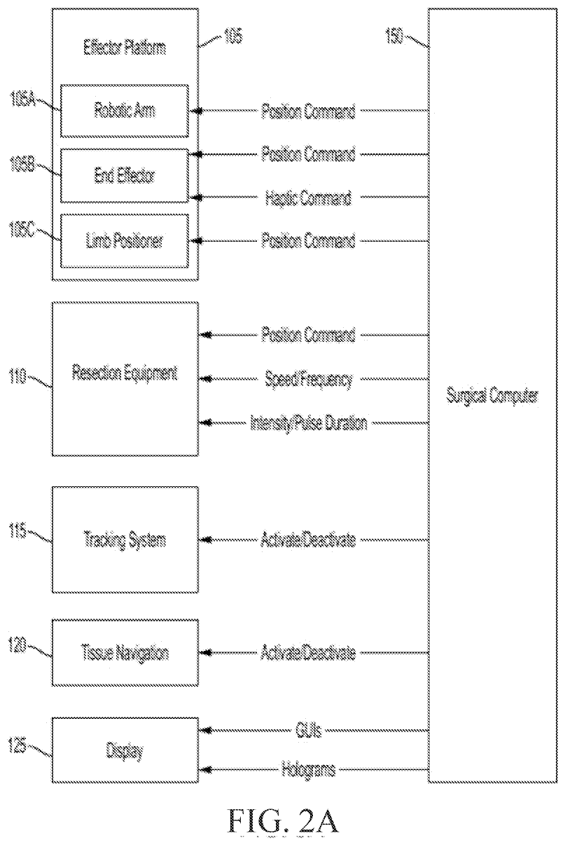

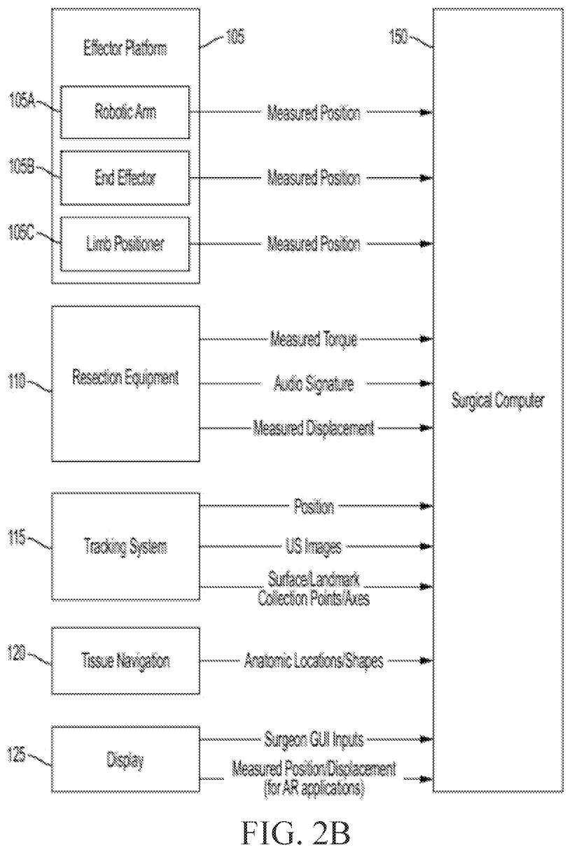

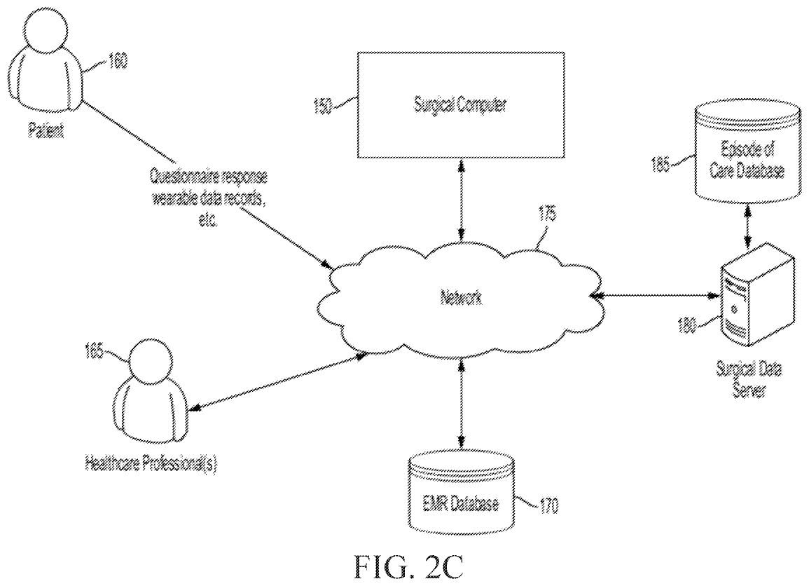

Furthermore, the data can be used for educational, training, or research purposes. For example, using the network-based approach described below in FIG. 2C, other doctors or students can remotely view surgeries in interfaces that allow them to selectively view data as it is collected from the various components of the CASS 100. After the surgical procedure, similar interfaces may be used to “playback” a surgery for training or other educational purposes, or to identify the source of any issues or complications with the procedure.

Data acquired during the pre-operative phase generally includes all information collected or generated prior to the surgery. Thus, for example, information about the patient may be acquired from a patient intake form or electronic medical record (EMR). Examples of patient information that may be collected include, without limitation, patient demographics, diagnoses, medical histories, progress notes, vital signs, medical history information, allergies, and lab results. The pre-operative data may also include images related to the anatomical area of interest. These images may be captured, for example, using Magnetic Resonance Imaging (MRI), Computed Tomography (CT), X-ray, ultrasound, or any other modality known in the art. The pre-operative data may also comprise quality of life data captured from the patient. For example, in one embodiment, pre-surgery patients use a mobile application (“app”) to answer questionnaires regarding their current quality of life. In some embodiments, preoperative data used by the CASS 100 includes demographic, anthropometric, cultural, or other specific traits about a patient that can coincide with activity levels and specific patient activities to customize the surgical plan to the patient. For example, certain cultures or demographics may be more likely to use a toilet that requires squatting on a daily basis.

FIGS. 2A and 2B provide examples of data that may be acquired during the intra-operative phase of an episode of care. These examples are based on the various components of the CASS 100 described above with reference to FIG. 1; however, it should be understood that other types of data may be used based on the types of equipment used during surgery and their use.

FIG. 2A shows examples of some of the control instructions that the Surgical Computer 150 provides to other components of the CASS 100, according to some embodiments. Note that the example of FIG. 2A assumes that the components of the Effector Platform 105 are each controlled directly by the Surgical Computer 150. In embodiments where a component is manually controlled by the Surgeon 111, instructions may be provided on the Display 125 or AR HMD 155 instructing the Surgeon 11 1 how to move the component.

The various components included in the Effector Platform 105 are controlled by the Surgical Computer 150 providing position commands that instruct the component where to move within a coordinate system. In some embodiments, the Surgical Computer 150 provides the Effector Platform 105 with instructions defining how to react when a component of the Effector Platform 105 deviates from a surgical plan. These commands are referenced in FIG. 2A as “haptic” commands. For example, the End Effector 105B may provide a force to resist movement outside of an area where resection is planned. Other commands that may be used by the Effector Platform 105 include vibration and audio cues.

In some embodiments, the end effectors 105B of the robotic arm 105A are operatively coupled with cutting guide 105D. In response to an anatomical model of the surgical scene, the robotic arm 105A can move the end effectors 105B and the cutting guide 105D into position to match the location of the femoral or tibial cut to be performed in accordance with the surgical plan. This can reduce the likelihood of error, allowing the vision system and a processor utilizing that vision system to implement the surgical plan to place a cutting guide 105D at the precise location and orientation relative to the tibia or femur to align a cutting slot of the cutting guide with the cut to be performed according to the surgical plan. Then, a surgeon can use any suitable tool, such as an oscillating or rotating saw or drill to perform the cut (or drill a hole) with perfect placement and orientation because the tool is mechanically limited by the features of the cutting guide 105D. In some embodiments, the cutting guide 105D may include one or more pin holes that are used by a surgeon to drill and screw or pin the cutting guide into place before performing a resection of the patient tissue using the cutting guide. This can free the robotic arm 105A or ensure that the cutting guide 105D is fully affixed without moving relative to the bone to be resected. For example, this procedure can be used to make the first distal cut of the femur during a total knee arthroplasty. In some embodiments, where the arthroplasty is a hip arthroplasty, cutting guide 105D can be fixed to the femoral head or the acetabulum for the respective hip arthroplasty resection. It should be understood that any arthroplasty that utilizes precise cuts can use the robotic arm 105A and/or cutting guide 105D in this manner.

The Resection Equipment 110 is provided with a variety of commands to perform bone or tissue operations. As with the Effector Platform 105, position information may be provided to the Resection Equipment 110 to specify where it should be located when performing resection. Other commands provided to the Resection Equipment 110 may be dependent on the type of resection equipment. For example, for a mechanical or ultrasonic resection tool, the commands may specify the speed and frequency of the tool. For Radiofrequency Ablation (RFA) and other laser ablation tools, the commands may specify intensity and pulse duration.

Some components of the CASS 100 do not need to be directly controlled by the Surgical Computer 150; rather, the Surgical Computer 150 only needs to activate the component, which then executes software locally specifying the manner in which to collect data and provide it to the Surgical Computer 150. In the example of FIG. 2A, there are two components that are operated in this manner; the Tracking System 115 and the Tissue Navigation System 120.

The Surgical Computer 150 provides the Display 125 with any visualization that is needed by the Surgeon 111 during surgery. For monitors, the Surgical Computer 150 may provide instructions for displaying images, GUIs, etc. using techniques known in the art. The display 125 can include various portions of the workflow of a surgical plan. During the registration process, for example, the display 125 can show a preoperatively constructed 3D bone model and depict the locations of the probe as the surgeon uses the probe to collect locations of anatomical landmarks on the patient. The display 125 can include information about the surgical target area. For example, in connection with a TKA, the display 125 can depict the mechanical and anatomical axes of the femur and tibia. The display 125 can depict varus and valgus angles for the knee joint based on a surgical plan, and the CASS 100 can depict how such angles will be affected if contemplated revisions to the surgical plan are made. Accordingly, the display 125 is an interactive interface that can dynamically update and display how changes to the surgical plan would impact the procedure and the final position and orientation of implants installed on bone.

As the workflow progresses to preparation of bone cuts or resections, the display 125 can depict the planned or recommended bone cuts before any cuts are performed. The surgeon 111 can manipulate the image display to provide different anatomical perspectives of the target area and can have the option to alter or revise the planned bone cuts based on intraoperative evaluation of the patient. The display 125 can depict how the chosen implants would be installed on the bone if the planned bone cuts are performed. If the surgeon 111 choses to change the previously planned bone cuts, the display 125 can depict how the revised bone cuts would change the position and orientation of the implant when installed on the bone.

The display 125 can provide the surgeon 111 with a variety of data and information about the patient, the planned surgical intervention, and the implants. Various patient-specific information can be displayed, including real-time data concerning the patient's health such as heart rate, blood pressure, etc. The display 125 also can include information about the anatomy of the surgical target region including the location of landmarks, the current state of the anatomy (e.g., whether any resections have been made, the depth and angles of planned and executed bone cuts), and future states of the anatomy as the surgical plan progresses. The display 125 also can provide or depict additional information about the surgical target region. For a TKA, the display 125 can provide information about the gaps (e.g., gap balancing) between the femur and tibia and how such gaps will change if the planned surgical plan is carried out. For a TKA, the display 125 can provide additional relevant information about the knee joint such as data about the joint's tension (e.g., ligament laxity) and information concerning rotation and alignment of the joint. The display 125 can depict how the planned implants'locations and positions will affect the patient as the knee joint is flexed. The display 125 can depict how the use of different implants or the use of different sizes of the same implant will affect the surgical plan and preview how such implants will be positioned on the bone. The CASS 100 can provide such information for each of the planned bone resections in a TKA or THA. In a TKA, the CASS 100 can provide robotic control for one or more of the planned bone resections. For example, the CASS 100 can provide robotic control only for the initial distal femur cut, and the surgeon 111 can manually perform other resections (anterior, posterior and chamfer cuts) using conventional means, such as a 4-in-1 cutting guide or jig 105D.

The display 125 can employ different colors to inform the surgeon of the status of the surgical plan. For example, un-resected bone can be displayed in a first color, resected bone can be displayed in a second color, and planned resections can be displayed in a third color. Implants can be superimposed onto the bone in the display 125, and implant colors can change or correspond to different types or sizes of implants.

The information and options depicted on the display 125 can vary depending on the type of surgical procedure being performed. Further, the surgeon 111 can request or select a particular surgical workflow display that matches or is consistent with his or her surgical plan preferences. For example, for a surgeon Ill who typically performs the tibial cuts before the femoral cuts in a TKA, the display 125 and associated workflow can be adapted to take this preference into account. The surgeon 111 also can preselect that certain steps be included or deleted from the standard surgical workflow display, For example, if a surgeon 111 uses resection measurements to finalize an implant plan but does not analyze ligament gap balancing when finalizing the implant plan, the surgical workflow display can be organized into modules, and the surgeon can select which modules to display and the order in which the modules are provided based on the surgeon's preferences or the circumstances of a particular surgery. Modules directed to ligament and gap balancing, for example, can include pre-and post-resection ligament/gap balancing, and the surgeon 111 can select which modules to include in their default surgical plan workflow depending on whether they perform such ligament and gap balancing before or after (or both) bone resections are performed.

For more specialized display equipment, such as AR HMDs, the Surgical Computer 150 may provide images, text, etc. using the data format supported by the equipment. For example, if the Display 125 is a holography device such as the Microsoft HoloLens™ or Magic Leap One™, the Surgical Computer 150 may use the HoloLens Application Program Interface (API) to send commands specifying the position and content of holograms displayed in the field of view of the Surgeon 111.

In some embodiments, one or more surgical planning models may be incorporated into the CASS 100 and used in the development of the surgical plans provided to the surgeon 111. The term “surgical planning model” refers to software that simulates the biomechanics performance of anatomy under various scenarios to determine the optimal way to perform cutting and other surgical activities. For example, for knee replacement surgeries, the surgical planning model can measure parameters for functional activities, such as deep knee bends, gait, etc., and select cut locations on the knee to optimize implant placement.

One example of a surgical planning model is the LIFEMOD™ simulation software from SMITH AND NEPHEW, INC. In some embodiments, the Surgical Computer 150 includes computing architecture that allows full execution of the surgical planning model during surgery (e.g., a GPU-based parallel processing environment). In other embodiments, the Surgical Computer 150 may be connected over a network to a remote computer that allows such execution, such as a Surgical Data Server 180 (see FIG. 2C). As an alternative to full execution of the surgical planning model, in some embodiments, a set of transfer functions are derived that simplify the mathematical operations captured by the model into one or more predictor equations. Then, rather than execute the full simulation during surgery, the predictor equations are used. Further details on the use of transfer functions are described in WIPO Publication No. 2020/037308, filed Aug. 19, 2019, entitled “Patient Specific Surgical Method and System,” the entirety of which is incorporated herein by reference.

FIG. 2B shows examples of some of the types of data that can be provided to the Surgical Computer 150 from the various components of the CASS 100. In some embodiments, the components may stream data to the Surgical Computer 150 in real-time or near real-time during surgery. In other embodiments, the components may queue data and send it to the Surgical Computer 150 at set intervals (e.g., every second). Data may be communicated using any format known in the art. Thus, in some embodiments, the components all transmit data to the Surgical Computer 150 in a common format. In other embodiments, each component may use a different data format, and the Surgical Computer 150 is configured with one or more software applications that enable translation of the data.

In general, the Surgical Computer 150 may serve as the central point where CASS data is collected. The exact content of the data will vary depending on the source. For example, each component of the Effector Platform 105 provides a measured position to the Surgical Computer 150. Thus, by comparing the measured position to a position originally specified by the Surgical Computer 150 (see FIG. 2B), the Surgical Computer can identify deviations that take place during surgery.

The Resection Equipment 110 can send various types of data to the Surgical Computer 150 depending on the type of equipment used. Example data types that may be sent include the measured torque, audio signatures, and measured displacement values. Similarly, the Tracking Technology 115 can provide different types of data depending on the tracking methodology employed. Example tracking data types include position values for tracked items (e.g., anatomy, tools, etc.), ultrasound images, and surface or landmark collection points or axes. The Tissue Navigation System 120 provides the Surgical Computer 150 with anatomic locations, shapes, etc. as the system operates.

Although the Display 125 generally is used for outputting data for presentation to the user, it may also provide data to the Surgical Computer 150. For example, for embodiments where a monitor is used as part of the Display 125, the Surgeon 111 may interact with a GUI to provide inputs which are sent to the Surgical Computer 150 for further processing. For AR applications, the measured position and displacement of the HMD may be sent to the Surgical Computer 150 so that it can update the presented view as needed.

During the post-operative phase of the episode of care, various types of data can be collected to quantify the overall improvement or deterioration in the patient's condition as a result of the surgery. The data can take the form of, for example, self-reported information reported by patients via questionnaires. For example, in the context of a knee replacement surgery, functional status can be measured with an Oxford Knee Score questionnaire, and the post-operative quality of life can be measured with a EQ5D-5L questionnaire. Other examples in the context of a hip replacement surgery may include the Oxford Hip Score, Harris Hip Score, and WOMAC (Western Ontario and McMaster Universities Osteoarthritis index). Such questionnaires can be administered, for example, by a healthcare professional directly in a clinical setting or using a mobile app that allows the patient to respond to questions directly. In some embodiments, the patient may be outfitted with one or more wearable devices that collect data relevant to the surgery. For example, following a knee surgery, the patient may be outfitted with a knee brace that includes sensors that monitor knee positioning, flexibility, etc. This information can be collected and transferred to the patient's mobile device for review by the surgeon to evaluate the outcome of the surgery and address any issues. In some embodiments, one or more cameras can capture and record the motion of a patient's body segments during specified activities postoperatively. This motion capture can be compared to a biomechanics model to better understand the functionality of the patient's joints and better predict progress in recovery and identify any possible revisions that may be needed.

The post-operative stage of the episode of care can continue over the entire life of a patient. For example, in some embodiments, the Surgical Computer 150 or other components comprising the CASS 100 can continue to receive and collect data relevant to a surgical procedure after the procedure has been performed. This data may include, for example, images, answers to questions, “normal” patient data (e.g., blood type, blood pressure, conditions, medications, etc.), biometric data (e.g., gait, etc.), and objective and subjective data about specific issues (e.g., knee or hip joint pain). This data may be explicitly provided to the Surgical Computer 150 or other CASS component by the patient or the patient's physician(s). Alternatively or additionally, the Surgical Computer 150 or other CASS component can monitor the patient's EMR and retrieve relevant information as it becomes available. This longitudinal view of the patient's recovery allows the Surgical Computer 150 or other CASS component to provide a more objective analysis of the patient's outcome to measure and track success or lack of success for a given procedure. For example, a condition experienced by a patient long after the surgical procedure can be linked back to the surgery through a regression analysis of various data items collected during the episode of care. This analysis can be further enhanced by performing the analysis on groups of patients that had similar procedures and/or have similar anatomies.

In some embodiments, data is collected at a central location to provide for easier analysis and use. Data can be manually collected from various CASS components in some instances. For example, a portable storage device (e.g., USB stick) can be attached to the Surgical Computer 150 into order to retrieve data collected during surgery. The data can then be transferred, for example, via a desktop computer to the centralized storage. Alternatively, in some embodiments, the Surgical Computer 150 is connected directly to the centralized storage via a Network 175 as shown in FIG. 2C.

FIG. 2C illustrates a “cloud-based” implementation in which the Surgical Computer 150 is connected to a Surgical Data Server 180 via a Network 175. This Network 175 may be, for example, a private intranet or the Internet. In addition to the data from the Surgical Computer 150, other sources can transfer relevant data to the Surgical Data Server 180. The example of FIG. 2C shows three additional data sources: the Patient 160, Healthcare Professional(s) 165, and an EMR Database 170. Thus, the Patient 160 can send pre-operative and post-operative data to the Surgical Data Server 180, for example, using a mobile app. The Healthcare Professional(s) 165 includes the surgeon and his or her staff as well as any other professionals working with Patient 160 (e.g., a personal physician, a rehabilitation specialist, etc.). It should also be noted that the EMR Database 170 may be used for both pre-operative and post-operative data. For example, assuming that the Patient 160 has given adequate permissions, the Surgical Data Server 180 may collect the EMR of the Patient pre-surgery. Then, the Surgical Data Server 180 may continue to monitor the EMR for any updates post-surgery.

At the Surgical Data Server 180, an Episode of Care Database 185 is used to store the various data collected over a patient's episode of care. The Episode of Care Database 185 may be implemented using any technique known in the art. For example, in some embodiments, a SQL-based database may be used where all of the various data items are structured in a manner that allows them to be readily incorporated in two SQL's collection of rows and columns. However, in other embodiments a No-SQL database may be employed to allow for unstructured data, while providing the ability to rapidly process and respond to queries. As is understood in the art, the term “No-SQL” is used to define a class of data stores that are non-relational in their design. Various types of No-SQL databases may generally be grouped according to their underlying data model. These groupings may include databases that use column-based data models (e.g., Cassandra), document-based data models (e.g., MongoDB), key-value based data models (e.g., Redis), and/or graph-based data models (e.g., Allego). Any type of No-SQL database may be used to implement the various embodiments described herein and, in some embodiments, the different types of databases may support the Episode of Care Database 185.

Data can be transferred between the various data sources and the Surgical Data Server 180 using any data format and transfer technique known in the art. It should be noted that the architecture shown in FIG. 2C allows transmission from the data source to the Surgical Data Server 180, as well as retrieval of data from the Surgical Data Server 180 by the data sources. For example, as explained in detail below, in some embodiments, the Surgical Computer 150 may use data from past surgeries, machine learning models, etc. to help guide the surgical procedure.

In some embodiments, the Surgical Computer 150 or the Surgical Data Server 180 may execute a de-identification process to ensure that data stored in the Episode of Care Database 185 meets Health Insurance Portability and Accountability Act (HIPAA) standards or other requirements mandated by law. HIPAA provides a list of certain identifiers that must be removed from data during de-identification. The aforementioned de-identification process can scan for these identifiers in data that is transferred to the Episode of Care Database 185 for storage. For example, in one embodiment, the Surgical Computer 150 executes the de-identification process just prior to initiating transfer of a particular data item or set of data items to the Surgical Data Server 180. In some embodiments, a unique identifier is assigned to data from a particular episode of care to allow for re-identification of the data if necessary.

Although FIGS. 2A-C discuss data collection in the context of a single episode of care, it should be understood that the general concept can be extended to data collection from multiple episodes of care. For example, surgical data may be collected over an entire episode of care each time a surgery is performed with the CASS 100 and stored at the Surgical Computer 150 or at the Surgical Data Server 180. As explained in further detail below, a robust database of episode of care data allows the generation of optimized values, measurements, distances, or other parameters and other recommendations related to the surgical procedure. In some embodiments, the various datasets are indexed in the database or other storage medium in a manner that allows for rapid retrieval of relevant information during the surgical procedure. For example, in one embodiment, a patient-centric set of indices may be used so that data pertaining to a particular patient or a set of patients similar to a particular patient can be readily extracted. This concept can be similarly applied to surgeons, implant characteristics, CASS component versions, etc.

Further details of the management of episode of care data is described in U.S. Ser. No. 16/847,183 , filed Apr. 13, 2020, published as U.S. Publication No. 2020/0243199, and entitled “METHODS AND SYSTEMS FOR PROVIDING AN EPISODE OF CARE,” the entirety of which is incorporated herein by reference.

### Patient-specific Operative Planning

The present disclosure is generally directed to systems and methods for planning patient-specific surgical procedures. In particular, the present disclosure describes systems and methods for allowing surgeons to select preferred or default alignment algorithms for a variety of different patient categories or types, which can then be automatically retrieved and applied to configure the surgical system without requiring the surgeon or surgical team to manually select and enter the alignment algorithm data. Further, the systems and methods described herein allow the surgeon to visualize comparisons between the different alignment algorithms for each patient category to aid in allowing the surgeon to select the default alignment algorithm for those patient categories based on the surgeon's preferences.

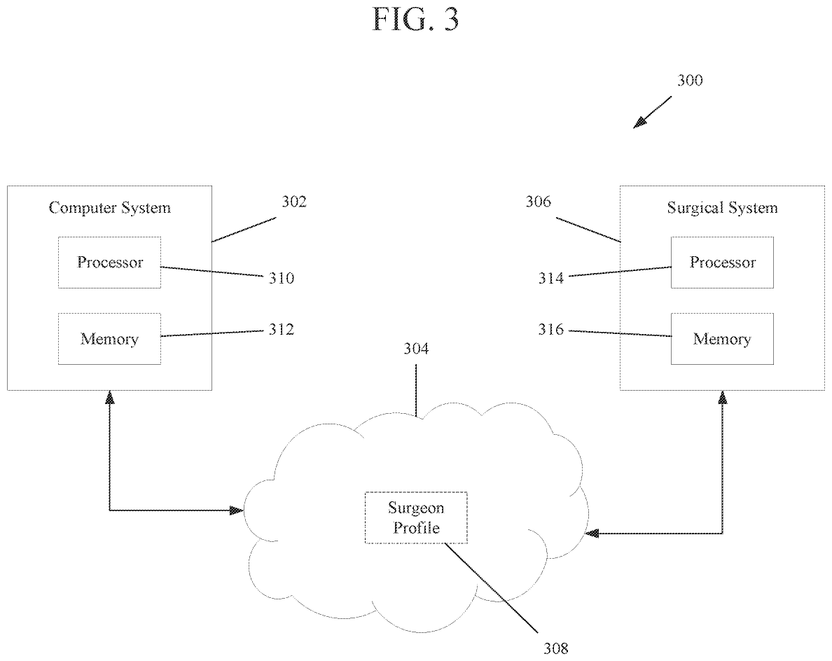

In order to facilitate the ability for surgical teams to provide surgical plans that are tailored for individual patients, the systems and methods described herein provide surgeons with the ability to make a customized profile that they can use to store their surgical planning preferences for various types of patient categories. In one embodiment, such as is shown in FIG. 3, a system 300 could include a computer system 302 that is coupled to a surgical system 306 via a network 304. In one embodiment, the surgical system 306 could include the surgical systems described above in connection with FIGS. 1-2C. The network 304 could include a cloud computing platform (e.g., Amazon Web Services). In one embodiment, the computer system 302 could be remote from the surgical system 306. In another embodiment, the computer system 302 could be integral to or a component of the surgical system 306. The computer system 302 could include a processor 310 and a memory 312. The surgical system 306 can likewise include a processor 314 and a memory 316. In one embodiment, a software application provided to end users (e.g., surgeons or surgical team members) could be implemented as a software-as-a-service (Saas) architecture that is hosted via the network 304.

In application, a surgeon can create a profile 308 that stores the surgeon's preferences for particular surgical procedures and/or patient types. This profile 308 can be downloaded or otherwise accessed by the surgical system 306 in connection with the performance of a surgical procedure in order to preconfigure the surgical system 306 to the surgeon's preference (as dictated by the surgeon profile 308). In one embodiment, the surgical system 306 could include a robotic or computer-assisted surgical system and the surgeon profile 308 can store various parameters or other inputs for configuring the surgical system 306.

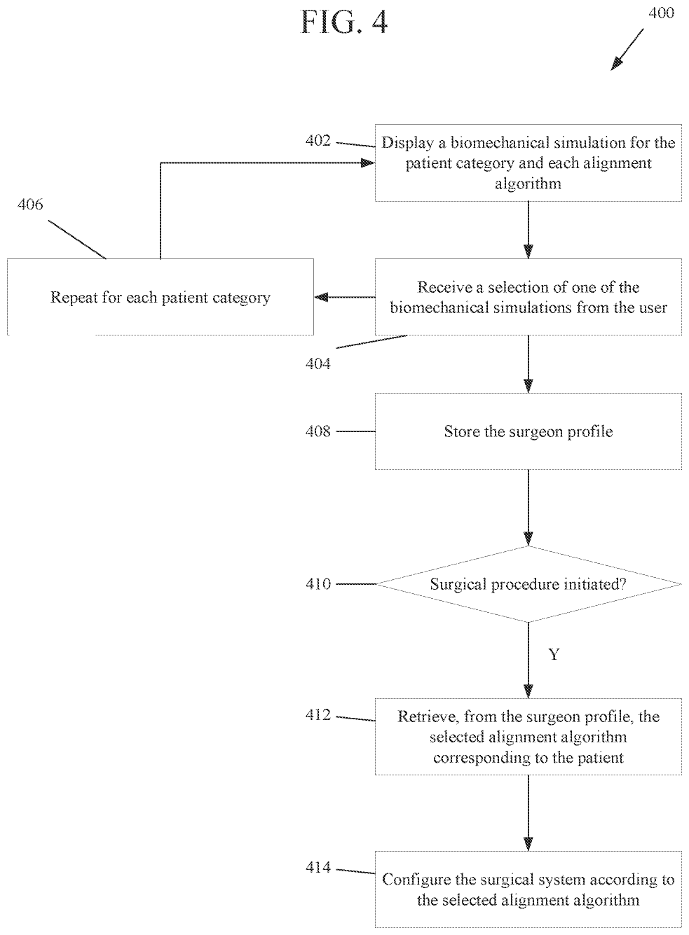

One embodiment of a process 400 for planning a patient-specific surgical procedure is shown in FIG. 4. In one embodiment, the process 400 can be embodied as instructions stored in a memory (e.g., the memory 312 and/or the memory 316) that, when executed by a processor (e.g., the processor 310 and/or the processor 314), cause the system 300 to perform the process 400. In various embodiments, the process 400 can be embodied as software, hardware, firmware, and various combinations thereof. In various embodiments, the process 400 can be executed by and/or between a variety of different devices or systems. For example, various combinations of steps of the process 400 could be executed by the computer system 302, the network 304, and/or the surgical system 306. In various embodiments, the system 300 executing the process 400 can utilize distributed processing, parallel processing, cloud processing, and/or edge computing techniques. The process 400 is described below as being executed by the system 300; accordingly, it should be understood that the functions can be individually or collectively executed by one or multiple devices or systems.

In operation, the system 300 allows the user (e.g., the surgeon) to preselect a preferred alignment algorithm for each of a series of defined patient categories. At the time of a surgical procedure, the system 300 can retrieve the appropriate alignment algorithm that corresponds to the category of the patient that is being operated on from the user's profile and preconfigure the surgical system 306 based on the retrieved alignment algorithm. In sum, the system 300 is designed to configure the surgical system 306 for each individual patient based on the surgeon's preselected preferences for that patient category, without requiring any additional action or selections by the surgeon or surgical team. Conventional surgical systems, particularly robotic surgical systems and computer-assisted surgical systems, need to be manually reconfigured for each individual patient. These conventional systems are disadvantageous because the surgeon and/or surgical team needs to review, analyze, and select a complicated series of parameters and algorithms for each individual surgical procedure, which is highly cumbersome and introduces the possibility for selection errors, Therefore, the system 300 described herein is beneficial because it allows the surgical system 306 to be automatically configured based on the surgeon's preselected preferences without requiring the cumbersome and error-prone manual configuration processes associated with conventional surgical systems. Further, the system 300 is beneficial because it allows users to visualize biomechanical simulations for each different alignment algorithm type and each patient category, rather than simply providing data or textually describing the consequences of each alignment algorithm. By displaying the results of each different alignment algorithm, the system 300 assists surgeons in understanding the consequences of each different alignment algorithm, which in turn assists them in selecting the alignment algorithm that they prefer for the patient type.

Accordingly, the system 300 executing the process 400 can display 402 a biomechanical simulation for the patient category and each alignment algorithm. The patient categories could include input variables generally used to configure robotic or computer-assisted surgical systems, such as the full leg alignment (e.g., varus/valgus angle) of the patient, the flexion-contracture degree of the patient, and the joint line of the patient. In some embodiments, the patient categories could further include various patient parameters, such as age, sex, height, weight, ethnicity, activity level, medical history, and surgical history. In some embodiments, the patient categories could include intraoperative surgical or patient data, such as ligament characterization, degree of arthritic degeneration or damage, joint laxity, joint gap, and/or joint tension. In some embodiments, the patient categories could include anatomical parameters, such as bone surface geometry or anatomical landmarks for sizing purposes or for characterization and classification. In some embodiments, the alignment algorithms could correspond to surgical techniques for controlling the positioning of the implant, including anatomic alignment, kinematic alignment, mechanical alignment, and physiological alignment. In some embodiments, the alignment algorithms could correspond to variables for various surgical procedures, including femur distal alignment (i.e., valgus rotation), femur distal resection, femur distal rotation (i.e., internal-external rotation), tibia proximal alignment (valgus rotation), tibia proximal resection, tibia proximal rotation (i.e., internal-external rotation), tibia proximal slope (i.e., flexion-extension rotation), femur between size selection, tibia between size selection, femur cut referencing (i.e., anterior or posterior), anterior cut preference, and max acceptable resection. In some embodiments, the alignment algorithms could include any combination of the aforementioned parameters, surgical variables, or other surgical system input parameters.

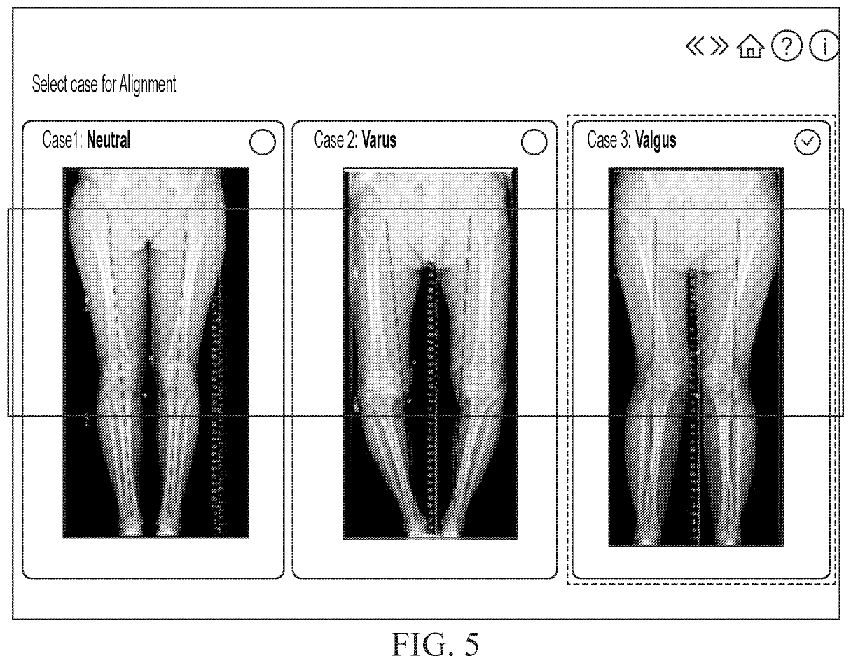

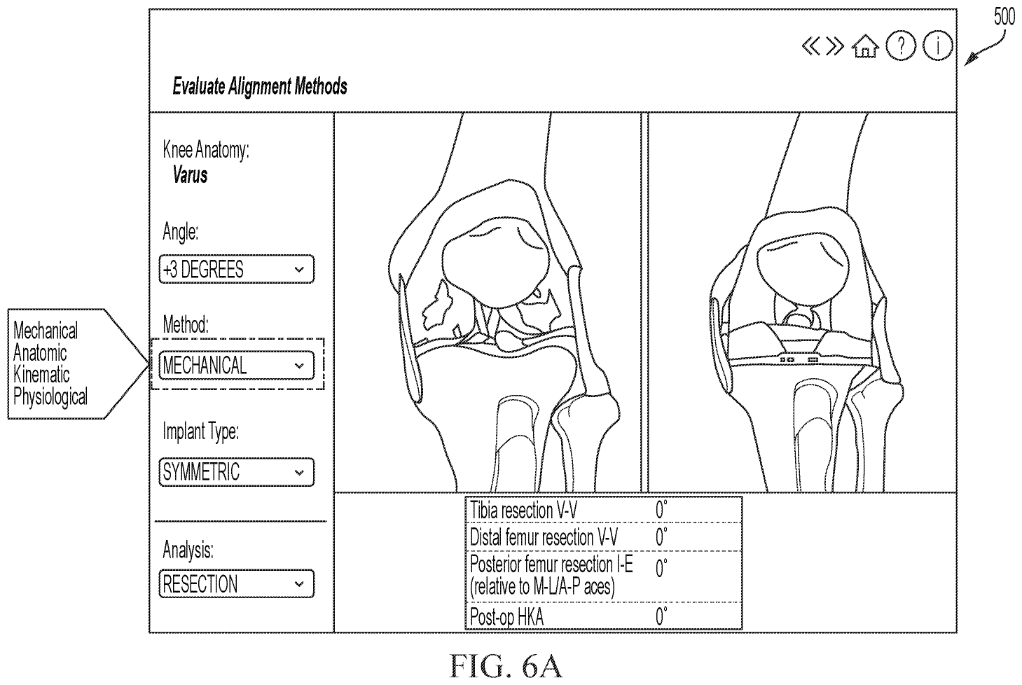

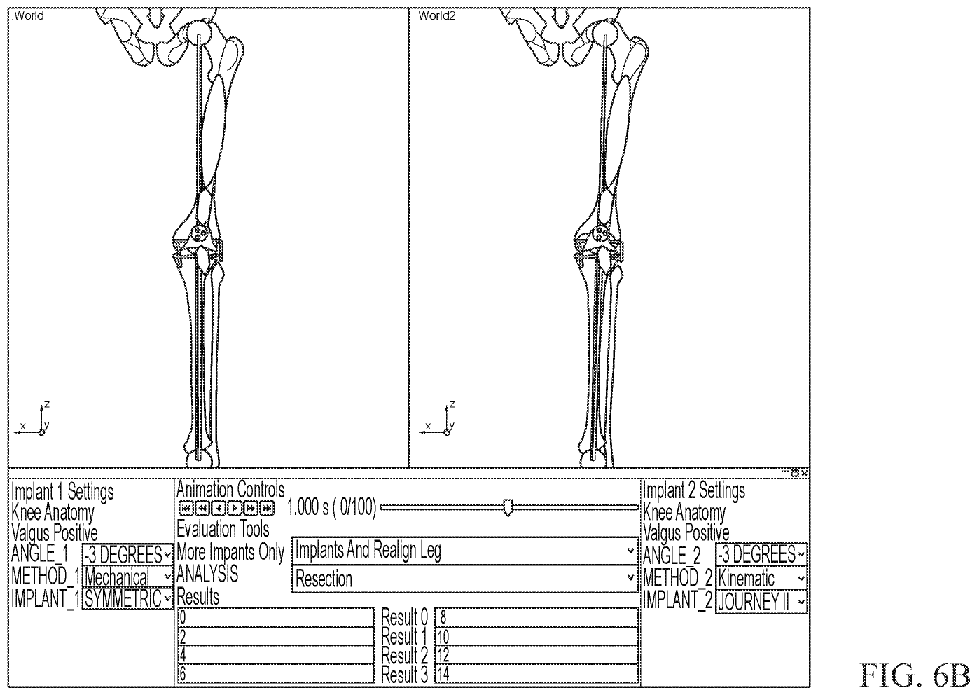

Therefore, as shown in FIG. 5, the system 300 can display each different patient category (varus, valgus, or neutral full leg alignment in this depicted embodiment). As shown in FIG. 6A, for each patient category, the system 300 can display 402 a biomechanical simulation 500 of each of the different alignment algorithm types for the selected implant type and the corresponding patient category (varus in this depicted embodiment). This allows the surgeon to visualize the effects of each of the different alignment algorithms for the particular patient category so that the surgeon can easily compare the alignment algorithms and select a preferred or default alignment algorithm for that particular patient category. In one embodiment, as shown in FIG. 6B, one or more of the biomechanical simulations corresponding to different alignment algorithms could further be displayed simultaneously to further assist surgeons in comparing the biomechanical effects of the different alignment algorithm options. Accordingly, the system 300 can receive 404 a selection of the surgeon's preferred alignment algorithm for the patient category.

In various embodiments, the biomechanical simulations can be performed and/or displayed 402 on the same or different computer systems. For example, the surgeon could utilize a computer system 302 (e.g., a laptop) to access a web or software-based application to visualize the biomechanical simulations for the patient categories and alignment algorithm types. Those biomechanical simulations could be performed by the computer system 302 itself or an external computer system that the computer system 302 is communicably coupled. In one embodiment, one or more of the patient measures (e.g., varus/valgus angle or flexion contracture) and/or the corresponding patient categories or subdivisions (e.g., threshold ranges for the various patient measures that define the patient categories) can be predetermined or hardcoded. In another embodiment, one or more of the patient measures and/or the patient categories could be selectable or definable by the user. In this embodiment, the biomechanical simulations could accordingly be updated for the selected patient measures and/or categories by, for example, drawing from a larger pre-executed simulation set or being performed on the fly (by the computer system 302 or another external computer system). Further, the user-selected patient measures and categories could be stored to the surgeon profile in the same manner as the selections made for the default or hardcoded patient measures and/or categories. In one embodiment, the alignment algorithms could be selectable or editable by the user. For example, a surgeon could select an initial alignment algorithm and then edit one or more parameters associated with the algorithm (e.g., resulting implant resection distance or angle). In this embodiment, the biomechanical simulations could accordingly be updated for the updated alignment algorithms by, for example, drawing from a larger pre-executed simulation set or being performed on the fly (by the computer system 302 or another external computer system). Further, the user-defined alignment algorithms could be stored to the surgeon profile in the same manner as the selections made for the default or hardcoded alignment algorithms. In yet another embodiment, both the patient measures (or the categories defined thereby) and the alignment algorithms could be selectable or editable by the user.

Further, the steps of displaying 402 the biomechanical simulations and receiving 404 a selection of a preferred alignment algorithm from the user can be repeated 406 for each patient category. Accordingly, the system 300 can receive the surgeon's preferred or default alignment algorithm for each of the defined patient categories and store 408 all of the selected alignment algorithms associated with the patient categories in a profile 308 associated with the surgeon. In other words, the surgeon profile 308 can define the default or preferred alignment algorithm that is used to preconfigure the surgical system 306 for each individual patient. The surgeon profile 308 can be stored in, for example, the network 304 to be later accessed by a surgical system 306. In one embodiment, the steps of displaying 402 biomechanical simulations and receiving 404 a selection from the user can further be repeated for multiple implant types.

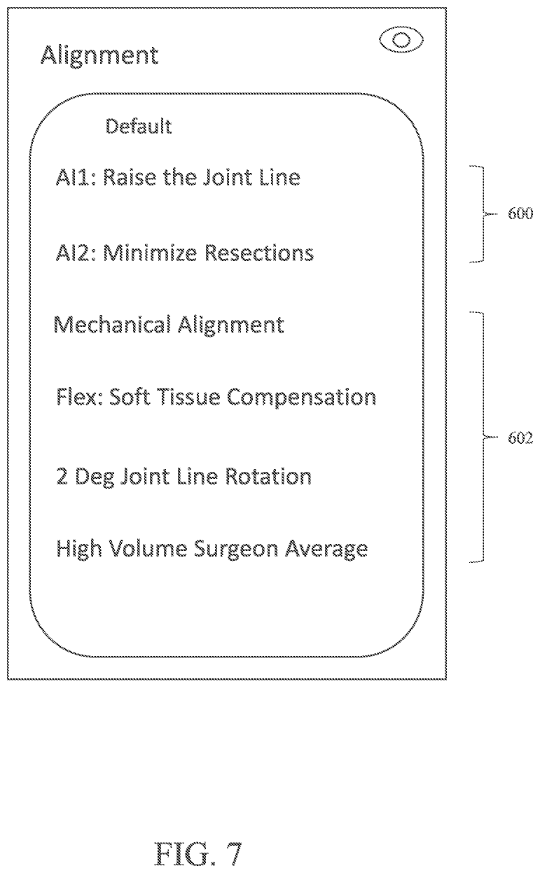

In some embodiments, the alignment algorithms could include manually defined algorithms 602 and AI-defined algorithms 600, as shown in FIG. 7. The manually defined algorithms 602 could be manually preprogrammed into the surgical system 306 for execution thereby. The Al-defined algorithms 600 can be clustered using supervised or unsupervised machine learning techniques from patient and surgical outcome data. In certain embodiments, the AI-defined algorithms 600 are clustered based on density, hierarchy, centroid, or distribution. Example machine learning techniques that can be employed include K-means clustering, iterative reducing and clustering using hierarchies, affinity propagation, distribution mixture models, spatial clustering, or mean-shift clustering.

Accordingly, the system 300 can determine 410 that a surgical procedure is being initiated using the surgical system 306 and access the profile 308 corresponding to the surgeon performing the procedure. In one embodiment, the surgeon can log into the surgical system 306 using login credentials and a password that is associated with the surgeon profile 306 so that the surgical system 306 can determine which surgeon is performing the procedure and retrieve the corresponding profile. Accordingly, the system 300 can retrieve 412 from the surgeon profile 308 the preselected alignment algorithm corresponding to (i) the patient category associated with the patient on which the procedure is being performed and (ii) the implant type that is being used in the surgical procedure. In some embodiments, patient data used to define the patient category can be determined preoperatively or intraoperatively. For example, patient data, such as age or weight, could be input into the surgical system 306 preoperatively (either by being manually input or being retrieved from the patient's electronic medical records). As another example, anatomical data, such as bone geometry, could be determined and input via preoperative imaging. As yet another example, surgical data, such as joint laxity or gap distance, could be input intraoperatively. Accordingly, the system 300 can configure 414 the surgical system 306 according to the alignment algorithm stored in the surgeon profile 308 for the patient category. For example, the surgical system 306 could change the initial position of the implant to reflect the preferred resection, preferred rotational adjustment, or combination of positioning parameters that were demonstrated biomechanically to provide a more favorable outcome for the encountered patient category.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art, all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

The functions and process steps herein may be performed automatically or wholly or partially in response to user command. An activity (including a step) performed automatically is performed in response to one or more executable instructions or device operation without user direct initiation of the activity.

1. A computer-implemented method for configuring a surgical system for performing a surgical procedure on a patient, the surgical procedure comprising a plurality of surgical parameters, the method comprising:
for each of a plurality of patient categories:
displaying a plurality of biomechanical simulations indicating postoperative performance of an implant, each of the plurality of biomechanical simulations corresponding to one of a plurality of alignment algorithms, and
receiving, from a user, a selection of one of the plurality of biomechanical simulations,
storing one of the plurality of alignment algorithms corresponding to each selection for each of the plurality of patient categories, thereby defining a user profile comprising a plurality of selected alignment algorithms corresponding to the plurality of patient categories; and
in response to the surgical system being used to perform the surgical procedure on the patient by the user:
retrieving, from the user profile, a selected alignment algorithm corresponding to a patient category of the plurality of patient categories with which the patient is associated, and
configuring the surgical system according to the selected alignment algorithm.

2. The method of claim 1, wherein the plurality of alignment algorithms are selected from the group consisting of anatomic alignment, kinematic alignment, mechanical alignment, and physiological alignment.

3. The method of claim 1, further comprising receiving a manual configuration for the plurality of alignment algorithms.

4. The method of claim 1, further comprising clustering, by a machine learning model, the plurality of alignment algorithms based on historical patient data.

5. The method of claim 1, wherein the surgical procedure is selected from the group consisting of a knee arthroplasty, a hip arthroplasty, and a shoulder arthroplasty.

6. The method of claim 1, wherein the surgical system comprises a robotic surgical system.

7. The method of claim 1, wherein the plurality of patient categories correspond to at least one of full leg varus/valgus angle, flexion-contracture degree, or joint line obliquity.

8. The method of claim 1, further comprising:
repeating the displaying of the plurality of biomechanical simulations indicating postoperative performance for a plurality of implant types; and
receiving, from a user, the selection of one of the plurality of biomechanical simulations for each of the plurality of implant types.

9. A system for performing a surgical procedure on a patient, the system comprising:
a computer system comprising a first processor and a first non-transitory memory, the first non-transitory memory storing first instructions that, when executed by the first processor, cause the computer system to:
for each of a plurality of patient categories:
display a plurality of biomechanical simulations indicating postoperative performance of an implant, each of the plurality of biomechanical simulations corresponding to one of a plurality of alignment algorithms, and
receive, from a user, a selection of one of the plurality of biomechanical simulations,
store one of the plurality of alignment algorithms corresponding to each selection for each of the plurality of patient categories, thereby defining a user profile comprising a plurality of selected alignment algorithms corresponding to the plurality of patient categories; and
a surgical system communicatively coupled to the computer system, the surgical system comprising a second processor and a second memory, the second non-transitory memory storing second instructions that, when executed by the second processor, cause the surgical system to:
in response to the surgical system being used to perform the surgical procedure on a patient by the user:
retrieve, from the user profile, a selected alignment algorithm corresponding to a patient category of the plurality of patient categories with which the patient is associated, and
configure the surgical system according to the selected alignment algorithm.

10. The system of claim 9, wherein the plurality of alignment algorithms are selected from the group consisting of anatomic alignment, kinematic alignment, mechanical alignment, and physiological alignment.

11. The system of claim 9, wherein the plurality of alignment algorithms are manually configured.

12. The system of claim 9, wherein the plurality of alignment algorithms are clustered by a machine learning model based on historical patient data 13. The system of claim 9, wherein the surgical procedure is selected from the group consisting of a knee arthroplasty, a hip arthroplasty, and a shoulder arthroplasty.

14. The system of claim 9, wherein the surgical system comprises a robotic surgical system.

15. The system of claim 9, wherein the plurality of patient categories correspond to at least one of full leg varus/valgus angle, flexion-contracture degree, or joint line obliquity.

16. The system of claim 9, wherein the first instructions, when executed by the first processor, further cause the computer system to:
repeat the displaying of the plurality of biomechanical simulations indicating postoperative performance for a plurality of implant types; and
receive, from a user, the selection of one of the plurality of biomechanical simulations for each of the plurality of implant types.

17. A system for performing a surgical procedure on a patient, the system comprising:
a computer system comprising a first processor and a first non-transitory memory, the first non-transitory memory storing first instructions that, when executed by the first processor, cause the computer system to:
train a machine learning model on a training set of patient outcome data;
cluster, by the machine learning model, a plurality of alignment algorithms;
for each of a plurality of patient categories:
display a plurality of biomechanical simulations indicating postoperative performance of an implant, each of the plurality of biomechanical simulations corresponding to one of the plurality of alignment algorithms, and
receive, from a user, a selection of one of the plurality of biomechanical simulations,
store one of the plurality of alignment algorithms corresponding to each selection for each of the plurality of patient categories, thereby defining a user profile comprising a plurality of selected alignment algorithms corresponding to the plurality of patient categories; and
a surgical system communicatively coupled to the computer system, the surgical system comprising a second processor and a second non-transitory memory, the second non-transitory memory storing second instructions that, when executed by the second processor, cause the surgical system to:
in response to the surgical system being used to perform the surgical procedure on a patient by the user:
retrieve, from the user profile, a selected alignment algorithm corresponding to a patient category of the plurality of patient categories with which the patient is associated, and
configure the surgical system according to the selected alignment algorithm.

18. The system of claim 17, wherein the machine learning model is supervised.

19. The system of claim 17, wherein the machine learning model is unsupervised.

20. The system of claim 17, further comprising a robotic surgical component;
wherein the second instructions, when executed by the second processor, further cause the surgical system to:
navigate the robotic surgical component according to the selected alignment algorithm.
